(19) United States
(12) Patent Application Publication
(10) Pub. No.: US 2026/0294382 A1
(43) Pub. Date: Oct. 1, 2026

(54) METHOD AND APPARATUS FOR ASSISTING AN ULTRASOUND OPERATOR

(71) Applicant: GE Precision Healthcare LLC, Waukesha, WI (US)

(72) Inventors: Nicholas Sleep, Cardiff (GB); Martin Benson, Cardiff (GB); Stephen Margetts, Cardiff (GB)

(21) Appl. No.: 19/479,309

(22) PCT Filed: Apr. 25, 2024
(86) PCT No.: PCT/GB2024/051085

§ 371 Date: Oct. 27, 2025

(30) Foreign Application Priority Data
Apr. 27, 2023 (GB) 2306231.8

(51) Int. Cl. A61B 8/08; A61B 8/00

(52) U.S. Cl. CPC A61B 8/085; A61B 8/463; A61B 8/5207; A61B 8/54

(57) ABSTRACT

A system for assisting an ultrasound operator that receives ultrasound image data from an input, analyses the data using an artificial intelligence unit which applies to the data a trained deep learning model to identify the probability that the image data represents a current scan site that is the desired scan site, and generates at least one of a number of types, depending on the calculated probability that the current site is the desired site.

### CROSS-REFERENCE TO RELATED APPLICATIONS

This application is the U.S. national stage entry under 35 U.S.C. § 371 of International Application No. PCT/GB2024/051085, filed on Apr. 25, 2024, which claims the benefit of UK Patent Application GB 2306231.8, filed on Apr. 27, 2023. The entire contents of the aforementioned applications are incorporated herein by reference.

### BACKGROUND

Ultrasound imaging is widely used in medical procedures, particularly for guiding regional anesthesia through peripheral nerve blocks. Accurate interpretation of ultrasound images is essential for identifying key anatomical structures such as nerves, arteries, and muscles. However, operators, especially those with limited experience, often face challenges in obtaining optimal views and correctly identifying these structures.

Existing systems, including those employing artificial intelligence, typically provide direct instructions to the operator regarding probe movement (e.g., “move left,” “tilt up,” “rotate right”). While these methods can assist in achieving the desired view, they are often complex and difficult to implement in practice, especially across a wide range of procedures and anatomical variations.

There remains a need for improved methods and apparatus that assist ultrasound operators in acquiring optimal anatomical views, particularly by providing intuitive guidance and real-time feedback, without relying on complicated movement instructions. Such solutions would enhance the accuracy and standardization of ultrasound image interpretation, supporting both expert and trainee users in performing peripheral nerve blocks and other ultrasound-guided procedures.

### SUMMARY

This summary introduces concepts that are described in more detail in the detailed description. It should not be used to identify essential features of the claimed subject matter, nor to limit the scope of the claimed subject matter.

According to one aspect of the present invention, there is provided an apparatus for assisting an ultrasound operator, the apparatus comprising a processor and an artificial intelligence unit, wherein the processor is arranged in use to receive one or more ultrasound images of a current scan site from an ultrasound probe, and to process the or each image using the artificial intelligence unit to make an assessment of the proximity of the current scan to a desired scan, wherein, based upon the assessment, the apparatus is arranged to present to the operator at least one of a number of different types of guidance prompt.

Proximity should be understood to include (though not be limited to) any of the following meanings: Distance in physical space, similarity of the anatomical features present within the image, similarity of the visual properties of the images.

Preferably, if the processor determines that the current scan site is below a first predetermined threshold proximity value of the desired scan site the apparatus presents to the operator a first type of guidance prompt.

Preferably, if the processor determines that the current scan site is greater than the first predetermined threshold proximity value of the desired scan site, but less than a second predetermined threshold proximity value of it, the apparatus presents to the operator a second type of guidance prompt.

Preferably, if the processor determines that the current scan site is greater than the second predetermined threshold proximity value of the desired scan site the apparatus presents to the operator a third type of guidance prompt.

The first type of prompt may comprise one or more of a short video sequence, an animation or a static image. This may be provided to the operator to provide hints on how the operator should place the probe during the scan.

The second type of prompt may comprise highlighting of one or more portions of the or each image, more preferably real-time highlighting, and/or at least one schematic image. This allows the operator to make small movements to fine-tune their image. It may also comprise displaying information that provides a quantitative measure of the proximity of the current scan site to the intended scan site. Finally, it may also include displaying information to the user to draw their attention to properties of the image that are currently lacking. For example, performing a nerve block may require that pressure is applied to the probe to collapse veins and the system may in this case, upon detecting that the veins are not collapsed, display a prompt to this effect to the user, in order that they can remedy it.

The third type of prompt may comprise providing a signal that the current scanning position is sufficiently close to the correct site for performing the intended procedure.

The prompts may be made using a display, which may be in addition to, or part of, a display used for displaying the or each scanned image. The prompts may include one or more of a visual prompt, an audible prompt or a haptic prompt.

According to another aspect of the present invention, there is provided a method of assisting an ultrasound operator, the method comprising receiving one or more ultrasound images of a current scan site from an ultrasound probe, processing the or each image using the artificial intelligence unit to make an assessment of the proximity of the current scan to a desired scan, wherein, based upon the assessment, the method comprises presenting to the operator at least one of a number of different types of guidance prompt.

Preferably, if the processing determines that the current scan site is below a first predetermined threshold proximity value of the desired scan site the method comprises presenting to the operator a first type of guidance prompt.

Preferably, if the processing determines that the current scan site is greater than the first predetermined threshold proximity value of the desired scan site, but less than a second predetermined threshold proximity value of it, the method comprises presenting to the operator a second type of guidance prompt.

Preferably, if the processing determines that the current scan site is greater than the second predetermined threshold proximity value of the desired scan site the method comprises presenting to the operator a third type of guidance prompt.

In a further aspect, the invention provides a computer program product on a computer readable medium, comprising instructions that, when executed by a computer, cause the computer to perform a method of assisting an ultrasound operator, the method comprising receiving one or more ultrasound images of a current scan site from an ultrasound probe, processing the or each image using the artificial intelligence unit to make an assessment of the proximity of the current scan to a desired scan, wherein, based upon the assessment, the method comprises presenting to the operator at least one of a number of different types of guidance prompt.

The invention also comprises a program for causing a device to perform a method of assisting an ultrasound operator, the method comprising receiving one or more ultrasound images of a current scan site from an ultrasound probe, processing the or each image using the artificial intelligence unit to make an assessment of the proximity of the current scan to a desired scan, wherein, based upon the assessment, the method comprises presenting to the operator at least one of a number of different types of guidance prompt.

The invention may include any combination of the features or limitations referred to herein, except such a combination of features as are mutually exclusive, or mutually inconsistent.

### BRIEF DESCRIPTION OF DRAWINGS

The present invention can be better understood by means of the description of the exemplary embodiments of the present invention in conjunction with the drawings, in which:

FIG. 1A shows schematically an image of an ultrasound scan including highlighted portions, being an example of a type of guidance prompt, according to an aspect;

FIG. 1B shows a schematic representation of part of a patient's anatomy, being another example of a type of guidance prompt, according to an aspect;

FIG. 2 shows schematically a further type of guidance prompt, according to an aspect;

FIG. 3 shows schematically an apparatus for assisting an operator according to an embodiment of the present invention, according to an aspect;

FIG. 4 shows schematically a method for assisting an operator according to an embodiment of the present invention, according to an aspect;

FIG. 5 shows schematically an optional further guidance prompt, according to an aspect; and

FIG. 6 shows schematically another optional guidance prompt, according to an aspect.

### DETAILED DESCRIPTION

The present invention relates to a method and an apparatus for assisting an operator during an ultrasound scan.

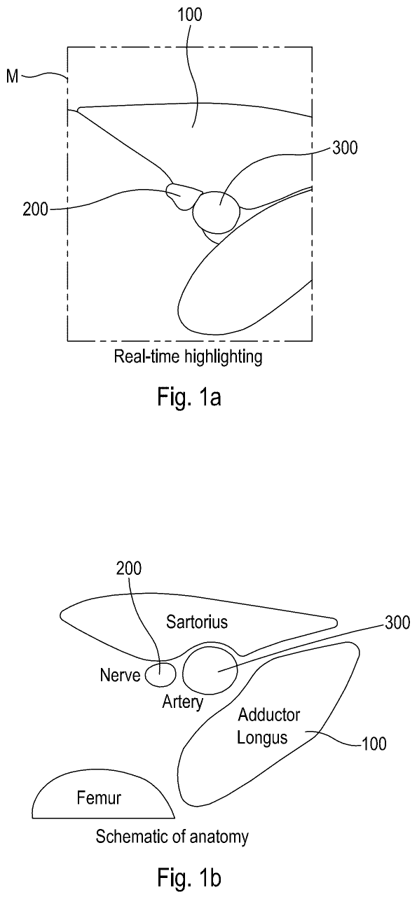

ScanNav™ Anatomy Peripheral Nerve Block (PNB) is a system designed to enhance the accuracy and standardization of ultrasound image interpretation by providing a color overlay of key sono-anatomical structures during a live ultrasound scan. The device is configured to provide real-time highlighting for ten commonly performed peripheral nerve blocks. FIG. 1a shows schematically an example of such a presentation, on a monitor M, in which features 100 (muscle), 200 (nerve) and 300 (artery) are highlighted in real time in the scan. It is intended to support users who are licensed to perform regional anesthesia but who are not confident. The device also incorporates reference material and training videos to help demonstrate the desired probe positioning for each supported procedure.

Anatomy PNB provides only indirect instructions to the operator about how to move the probe to obtain the optimum view. The operator is intended to obtain the ideal view by adjusting the probe to make the real-time highlighting of the key anatomical features match a schematic diagram. FIG. 1b is a schematic view of a patient's anatomy, as presented to the operator during a procedure, in which the same features shown in FIG. 1a are instead represented schematically.

Observation of trainees and experts during trial scans in which they were asked to locate the optimum view for a given procedure, has identified a key difference between their scanning techniques. The experts immediately placed the probe in the correct location (without reference to the ultrasound) and were thus able to see all the relevant anatomical features in their correct configuration. Following this, they made only very small movements towards optimizing their view.

The trainees usually placed the probe in a poor initial position, where the anatomical features they were looking for were either not in the right configuration or were not visible at all. From this point, they had to spend a long time moving the probe, trying to find some features they could recognize to help them improve their view. In addition, because they are starting from a poor initial position, the real-time highlighting provided by the tool is not as useful as when they are close to the optimum view, as there are fewer clues about how to move the probe.

There are previously considered systems that describe the use of AI to guide the operator to a desired view. These typically describe a system that gives the operator instructions about how to move the probe to obtain the desired view (e.g., “move left”, “tilt up”, “rotate right”). Such methods are complicated and difficult to implement, particularly in the general case.

Embodiments of the present invention aim to provide apparatus and a method for assisting an ultrasound operator, which aim to address the aforementioned problems.

In some implementations the present invention is concerned with helping an ultrasound operator locate a good initial view. The system uses an artificial intelligence (AI) unit to monitor the real-time ultrasound feed and assesses each image to determine the proximity of the current scan view to a desired target scan view, where “proximity” is as previously defined. If the current view is not in close proximity, a short video sequence (or possibly an animation or a static image) will be displayed to provide hints on how the operator should place the probe on the patient. If the current view is in close proximity to the desired view, the real-time highlighting will be shown along with the schematic, so that the operator can make small movements to fine-tune their image.

The computation of proximity of the current view to the desired view is performed using deep learning. A labelled image dataset is curated which contains “good” and “bad” examples of the target view, and standard deep learning techniques are used to turn this into a classifier. Given an image, the resulting trained model generates a probability that the input image is “good”. This probability is used to create a score for the current image.

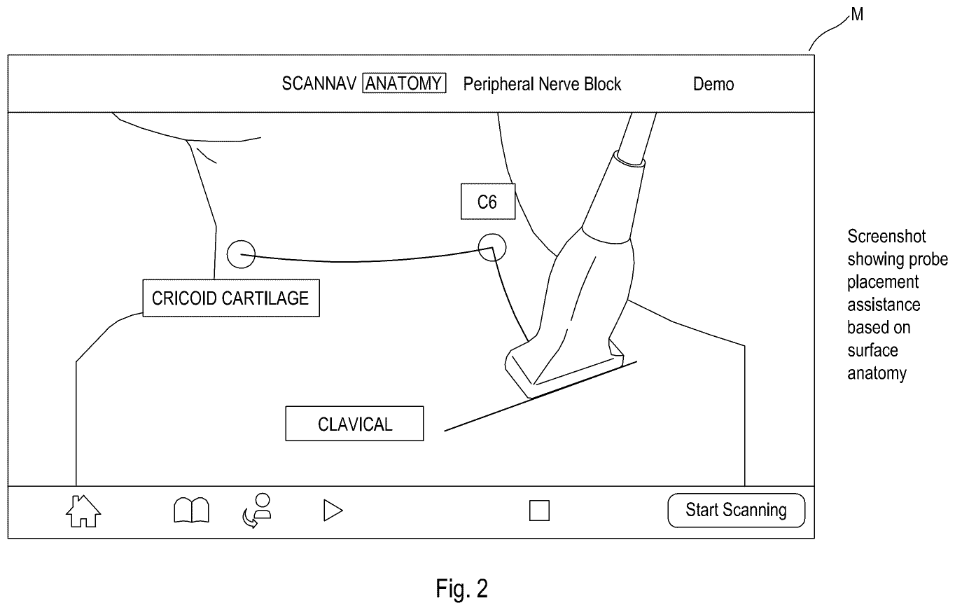

In a simple implementation, if the score is above a first given threshold but below a second threshold, the system will display the real-time highlighting and/or schematic anatomy, e.g. of the kind shown in FIGS. 1a and 1b. If it is below the first threshold, the system will display the probe location hints instead, e.g. of the kind shown on the monitor M in FIG. 2. Standard techniques for hysteresis control are applied to the scores to prevent the system from switching between the different display modes too rapidly.

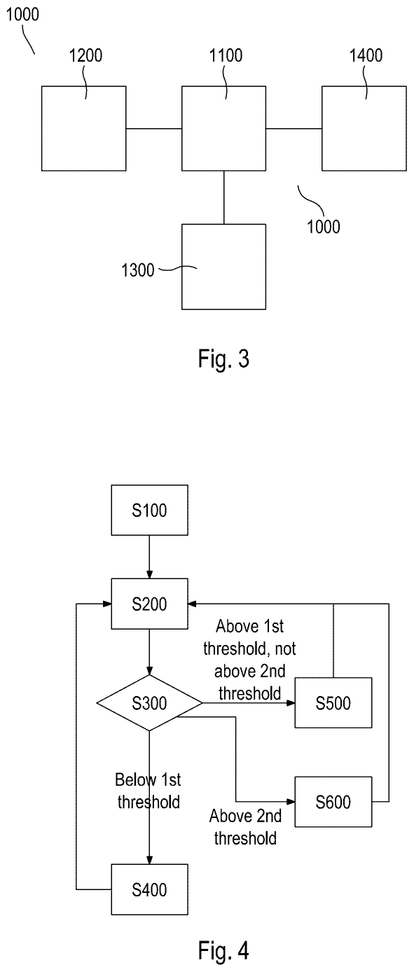

An embodiment of the apparatus according to the present invention is depicted schematically at 1000 in FIG. 3. The apparatus comprises a processor 1100 arranged to receive ultrasound image data from an input 1200. The processor 1100 analyses the data using an artificial intelligence unit 1300 which applies to the data a trained deep learning model to identify the probability that the image data represents a current scan site that is the desired scan site. An output 1400 is at least one of a number of types, depending on the calculated probability that the current site is the desired site. For example, a highlighted scan/schematic anatomy (as in FIG. 1a and/or FIG. 1b) is provided as a guidance prompt in the event that the current scan site is determined to be greater than a first predetermined proximity threshold, but less than a second threshold. Alternatively, the output 1400 may be one or more short video sequences, an animation, or a static image (as shown on monitor M in FIG. 2), in the event that the current scan site is determined to be less than a first predetermined threshold of proximity to the desired scan site. This is provided to the operator to give hints on how the operator should place the probe during the scan.

FIG. 4 shows schematically a method of assisting an ultrasound operator according to the above-described embodiment of the present invention. At Step S100, data of the current scan image is input to the processor. At Step S200 the processor computes a proximity probability score for the current scan image. At Step S300, a determination is made as to whether the score is (a) below a first predetermined score indicating a proximity value below a first predetermined threshold, (b) above the first threshold score but below a second threshold score, indicating a proximity that is greater than the first predetermined threshold but less than a second predetermined threshold, or (c) above a second predetermined score, indicating a proximity greater than the second predetermined threshold.

If the score is below the first threshold score, at Step S400 a first type of guidance is provided such as is shown in FIG. 2, i.e. a hint to the operator.

If the score is above a first threshold score but below a second threshold score, at Step S500, a second type of guidance prompt is provided, e.g. highlighting and/or schematic anatomy, for example as shown in FIG. 1a/1b.

If the determination is that the score is above the second predetermined threshold score, at Step S600, a third type of guidance is provided, for example being a signal or message that the current scanning location is considered to be at or near optimal, and therefore sufficiently proximal to the correct site for performing the intended procedure.

The image scores determined during analysis of the current scan images may need be smoothed by averaging over several frames, and the system may need to have some degree of hysteresis control to stop the display from flicking rapidly between the hint video and the real-time highlighting if the score is close to the threshold.

The system may optionally display the scores to the user, thus providing more direct feedback. This could be presented as a dial or bar chart.

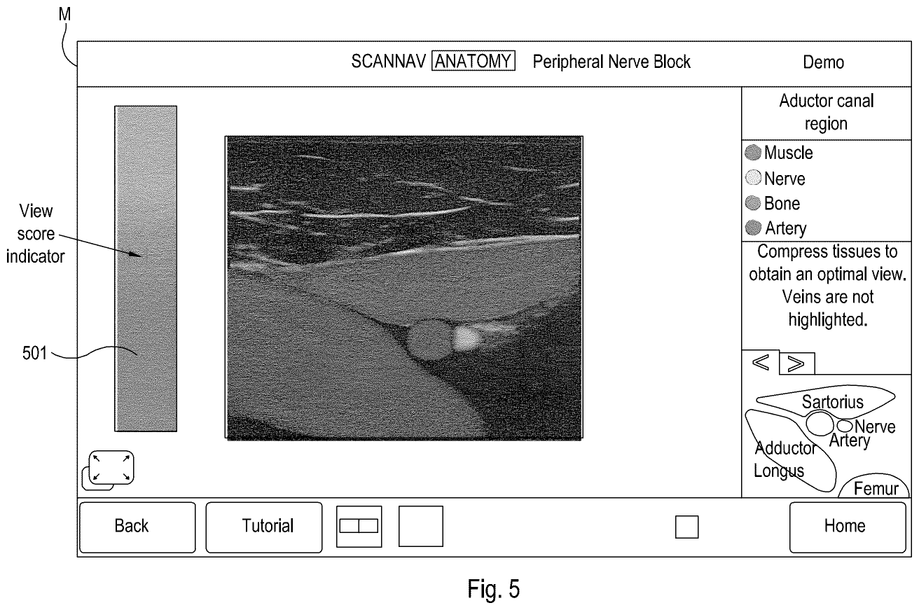

A further optional guidance prompt is shown in FIG. 5. When the score is above the aforementioned first threshold its value may additionally be displayed to the user in some form in order to provide feedback to them on whether or not they are approaching the intended scan site. In this case, the score is indicated in the monitor M in a view score indicator 501.

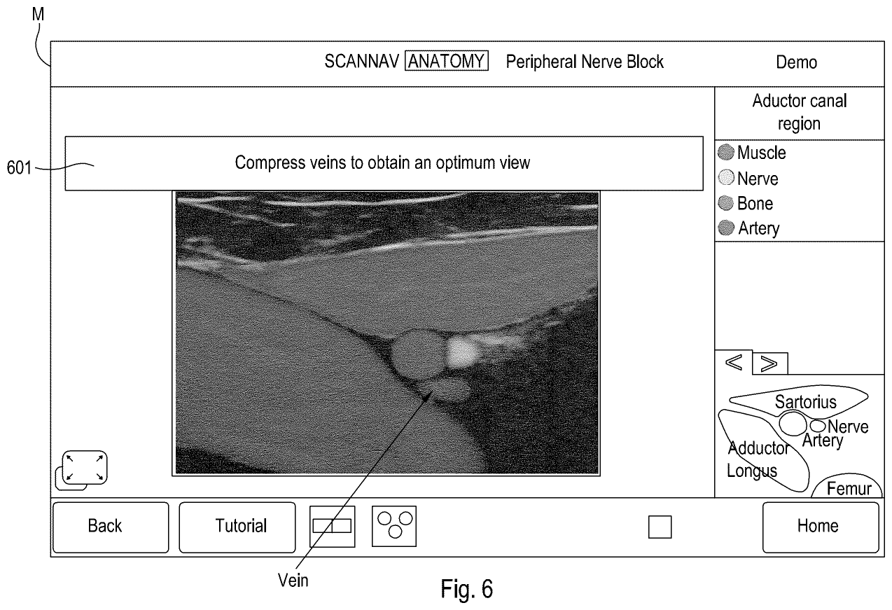

A still further optional guidance prompt is shown in FIG. 6. The system may also be configured to assess whether certain required properties of the image are present. For example, performing a nerve block may require that pressure is applied to the probe to collapse veins and the system may in this case, upon detecting that the veins are not collapsed, display a prompt to this effect to the user, in order that they can remedy it. Assessment of these properties may also be achieved through training a machine learning classifier on a training dataset of ultrasound images annotated for presence or absence of such properties. In this example, monitor M displays a prompt 601 to the user.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system for assisting an ultrasound operator, the apparatus comprising:
a memory storing instructions; and
a processor configured to execute the instructions to:
receive one or more ultrasound images of a current scan site from an ultrasound probe;
process at least one of the ultrasound images using the artificial intelligence unit to make an assessment of the proximity of the current scan to a desired scan; and
based upon the assessment, generate at least one of a number of different types of guidance prompt.

2. The system according to claim 1, wherein a first type of guidance prompt comprises one or more of a short video sequence, an animation or a static image.

3. The system according to claim 1, wherein a second type of guidance prompt comprises highlighting of one or more portions of the or each image.

4. The system according to claim 1, wherein a third type of guidance prompt comprises providing a signal that the current scanning position is sufficiently close to the correct site for performing an intended procedure.

5. The system according to claim 2, wherein, the processor is further configured to execute the instructions to generate a guidance prompt, of the at least one of the number of different types of guidance prompts, based on the processor determining that the current scan is less than a first predetermined threshold proximity of the desired scan.

6. The system according to claim 3, wherein the processor is further configured to execute the instructions to generate the second type of guidance prompt based on a determination that the current scan is greater than a first predetermined threshold proximity of the desired scan, but less than a second predetermined threshold proximity of it.

7. The system according to claim 4, wherein the processor is further configured to execute the instructions to generate the third type of guidance prompt based on a determination that that the current scan is greater than a second predetermined threshold proximity of the desired scan.

8. The system according to claim 1, wherein the processor is further configured to execute the instructions to calculate a probability score for each input image, which score relates to a probability that the image represents a current scan that is within/above a threshold proximity of a desired scan.

9. A method of assisting an ultrasound operator, the method comprising:
receiving one or more ultrasound images of a current scan site from an ultrasound probe,
processing the or each image to make an assessment of a proximity of the current scan to a desired scan; and
based upon the assessment, generate at least one of a number of different types of guidance prompt.

10. A non-transitory computer readable medium, storing instructions that, when executed by a computer, cause the computer to:
receive one or more ultrasound images of a current scan site from an ultrasound probe;
process at least one image to make an assessment of a proximity of the current scan to a desired scan, wherein, based upon the assessment, the method comprises presenting to the operator at least one of a number of different types of guidance prompt.
